(19) United States
(12) Patent Application Publication
(10) Pub. No.: US 2020/0197183 A1
(43) Pub. Date: Jun. 25, 2020

(54) SYSTEMS AND METHODS FOR DESIGNING AND MANUFACTURING ORTHOPEDIC JOINT REPLACEMENT IMPLANTS

(71) Applicant: David Scott NUTTER, Dayton, MD (US)

(72) Inventor: David Scott NUTTER, Dayton, MD (US)

(21) Appl. No.: 16/807,549

(22) Filed: Mar. 3, 2020

Related U.S. Application Data
(63) Continuation-in-part of application No. PCT/US2018/014432, filed on Jan. 19, 2018.
(60) Provisional application No. 62/528,315, filed on Jul. 3, 2017.

(51) Int. Cl. A61F 2/30; A61F 2/38; A61F 2/42; A61F 2/28

(52) U.S. Cl. CPC A61F 2/30942; A61F 2/3804; A61F 2/4202; A61F 2/2857; A61F 2002/30985; A61F 2002/3092; A61F 2002/4253; A61F 2002/4243; A61F 2002/4233; A61F 2002/30784; A61F 2002/4251

(57) ABSTRACT

Medical implants, specifically small to large orthopedic implant joint replacements. Systems and methods for manufacturing orthopedic implant joint replacements and their instrumentation.

### CROSS REFERENCE TO RELATED APPLICATIONS

This application is a continuation-in-part of PCT/US2018/14432 entitled SYSTEMS AND METHODS FOR DESIGNING AND MANUFACTURING ORTHOPEDIC JOINT REPLACEMENT IMPLANTS AND THEIR INSTRUMENTS and filed on Jan. 19, 2018, which claims priority to U.S. Provisional Application No. 62/528,315 entitled “First Metatarsal Phalangeal Total Titanium Total Joint Replacement” filed on Jul. 3, 2017. This application claims priority to U.S. Design Application No. 29/716,475 filed Dec. 10, 2019 entitled Anatomic Great Toe System. The contents of the above referenced applications are incorporated by reference in their entirety.

### FIELD OF THE INVENTION

The present invention is directed to medical implants, specifically small to large orthopedic implant joint replacements. The present invention is directed towards systems and methods for designing and manufacturing orthopedic implant joint replacements.

### BACKGROUND OF THE INVENTION

Joint replacement implants and their use on patients is a multi-billion-dollar industry. The problem with most orthopedic implant joint replacements, however, is that they are not made in a specific enough way to fit the natural biomechanical needs of a person's body. The current total joint replacements on the market are not manufactured in a very similar shape and form as they occur naturally in the body, in turn not being able to fully serve their best biomechanical purpose. Currently, orthopedic companies that make implants are essentially “free hand” drawing their implant designs, and making them in such a way that the implant designs are only just good enough to serve a joint's general ball and socket design purpose for most joints.

U.S. Pat. No. 8,353,965 is directed to small joint orthopedic implants and their manufacture. This patent is directed to a method for speedy manufacturing of a different joint replacement for each patient's specific anatomy. There are several drawbacks to this method. It is not practical, not affordable, and currently not being used to improve orthopedic implant manufacturing. The first major problem with U.S. Pat. No. 8,353,965 is that it would require a custom implant designed, manufactured and FDA approved for each patient, and an entire custom instrumentation set for that implant that would separately have to be FDA approved for each patient, costing the patient easily $625,000 to over $1,000,000 out of pocket depending on which joint they need replaced as well as having to wait up to 2 years to get their devices designed, manufactured, and to acquire the FDA approval for those custom devices.

U.S. Pat. No. 8,353,965 is also deficient since medical insurance companies might only cover 1-2% of the cost of making such an implant, a small fraction of the cost to make something custom for their patient.

The third major problem of U.S. Pat. No. 8,353,965 is that because of the allowed amount (the max amount that insurances allow a hospital or surgery center to bill them for each body parts' implant/surgery, which is in the range of $3,000-5,000 USD for small joint replacements such as fingers/toes and much more for the larger joint replacements), the recipient patients' insurance would only justify covering the cost of the surgery and not the cost of such an implant, leaving the patient completely out of pocket for the remaining amount, which could total hundreds of thousands of dollars or more.

A fourth problem of U.S. Pat. No. 8,353,965 is that the medical insurance company may consider the surgery to be cosmetic (as it relates to that one patient's specific bone shape) in which most medical insurance companies would not cover any of the cost.

All of the above reasons conclude why U.S. Pat. No. 8,353,965 is not being implemented/used to advance medical technology.

It is an object of the present invention to overcome these disadvantages set forth in U.S. Pat. No. 8,353,965. It is an object of the present invention to give a patient the best and most natural range of motion possible in the most affordable, efficient and practical way.

### SUMMARY OF THE INVENTION

It is an object of the invention to provide systems and methods for manufacturing, using and installing implantable joint replacements in patients.

It is an object of the invention to provide a cost effective and affordable way to make joint replacement implants that most closely relate to the human anatomy.

It is an object of the present invention to provide patients with anatomic shaped joint replacement implants in an efficient and affordable way.

It is an object of the invention to provide joint replacement implants in a variety of sizes to be made in advance (for example for 4-7 groups of people), similarly to how most implant sets are currently made.

It is an object of the invention to provide a method for manufacturing implant joint replacements modeling human bones.

It is an object of the present invention to provide one or more implants resulting from a method of manufacturing implant joint replacements modeling human bones.

It is an object of the invention to provide an instrumentation set of sizer instruments for measuring a joint that is to be resurfaced during surgery

It is an object of the present invention to provide the use of 3D laser scanning non-vivo human bones, the method allowing for artificial joints to be designed and manufactured in an identical shape and design to how they occur naturally in the body, to be used in joints such as the 1st metatarsal-phalangeal, lesser MPJ's, ankle, elbow, proximal metacarpophalangeal's, Trapezio-metacarpal thumb, and proximal interphalangeal joints. This will give the patient the most natural range of motion and the best, most anatomic joint possible.

It is an object of the present invention to provide a method of implant design/manufacture that is a much more possible, efficient, practical, precise and cost-effective method for manufacturing new implant's with little to no variation from the human bodies' natural biomechanical properties. This method is intended to re-define the way joint replacements are manufactured.

The inspiration to start this new method of production is from how bad some of the current implants are with the big toe total joint replacement for the metatarsal-phalangeal joint. One of the inventors has put many orthopedic joint replacements for the 1^{st} metatarsal-phalangeal joint and other toes in people to try to make their lives better, only to find that most of them didn't have the mechanical properties needed to prevent the bone from slipping off to the side and subluxing, and the ones that did were made out of silastic and wouldn't last any more than a few years to a decade or two at most without starting to disintegrate

The methods and systems of manufacture and use of the orthopedic implant joint replacements disclosed herein are intended to give the patient the best and most natural range of motion possible. This method may be used to improve many of the current hemi, and total joint replacements used on patients in orthopedic medicine.

Objects of the invention are achieved by providing a method for designing and manufacturing orthopedic joint replacement implants and their instrumentation modeling human bones to make anatomic shaped joint replacement implants that precisely replicate the human anatomy, the method comprising: securing human cadaver bones, securing 3D images, and/or radiographs of the joint of the human bone/bones from which the implant/implants are to be modeled after and creating joint replacements.

Objects of this invention are achieved by a method for manufacturing an anatomic shaped orthopedic joint replacement implant, the method comprising: providing an image source from a modeled bone selected from a group consisting of human bones, 3D images, laser scans, and/or radiographs of a joint of one or more human bones; acquiring and producing a solid part file or other form of three-dimensional (3D) CAD file based upon the image source; and using said file to create an orthopedic joint replacement implant.

In certain embodiments, the 3D computer file alters the image source to erase any imperfections to make smooth surfaces for the orthopedic joint replacement implant.

In certain embodiments, the 3D computer file is used with a 3D printer, CNC machine, or cast to create the orthopedic joint replacement implant.

In certain embodiments, non-vivo human bones are used as the model for the image source to design the orthopedic joint replacement implant.

In certain embodiments, the step of acquiring and producing the 3D computer file includes segregating the part of the bone where the joint resurfacing most commonly starts and saving the part of the bone into a new 3D computer file.

In certain embodiments, the orthopedic joint replacement implant includes a head.

In certain embodiments, the head is the perpendicular part of each implant that meets or connects to the stem, or the horizontal part of a “T” with the vertical part being the stem.

In certain embodiments, the head of the orthopedic joint replacement implant is affixed to the stem.

In certain embodiments, the head of the orthopedic joint replacement implant is affixed to an anchoring system that would anchor the artificial joint.

In certain embodiments, the orthopedic joint replacement implant is smoothed for the purpose of making very smooth surfaces for the head of the orthopedic joint replacement implant.

In certain embodiments, a designer alters a surface of the modeled bone, such as its cortical surface or surfaces, to erase any imperfections or deviations from the natural obvious contours of the one or more human bones.

Other objects of the invention are achieved by providing a method for a joint replacement implant in a patient, the method comprising: determining the location of a joint implant replacement on a patient; providing an image source from a modeled bone selected from a group consisting of human bones, 3D images of a joint of one or more cadaver bones; acquiring and producing a three-dimensional (3D) computer file based upon the image source; using the 3D computer file to create the orthopedic joint replacement implant, wherein the creation of the orthopedic joint replacement implant comprises: providing an indicator/marking to fabricate the implant, so the implant conforms to the dimensions of the one or more human bones, providing a stem/anchor to attach or fit on to the collar or head of the implant, providing a cap to fit on to the collar/head of the implant; and inserting the implant into the patient via a surgical procedure.

In certain embodiments, the one or more bones are cadaver bones.

In certain embodiments, the surgical procedure is an osteotomy.

Other objects of the invention are achieved by providing an anatomic shaped orthopedic joint replacement implant produced via the method described above, the anatomic shaped orthopedic joint replacement implant comprising: a stem/anchor to attach or fit on to the collar or head of the implant; and a cap to fit on to the collar/head of the implant.

In certain embodiments, the collar has an outer circumference that approximates the outer circumference of one modeled human bone, along with the head approximating the size and shape of the other modeled cadaver bone.

In certain embodiments, the collar is rotatable about the longitudinal axis of the implant with the head.

In certain embodiments, the implant that houses the cupping agent/bearing/cap is defined as the implant with a collar that rotates along the longitudinal axis of the implant with the head.

In certain embodiments, the stem or anchor is configured to be of varying sizes, and will sometimes increase incrementally and proportionally to the size of each implant.

Other objects of the invention are achieved by providing an anatomic shaped orthopedic joint replacement implant comprising: a collar having first and second opposing sides; a stem that projects from one side of the collar and which extends into the bone at the site of the osteotomy; and a cap/bearing that is connected to the post to complete the formation of the implant's head or base. For example, the cap/bearing on semi-constrained and non-constrained 1^{st} MTP total joint replacement attaches to the phalangeal base side.

In certain embodiments, the orthopedic joint replacement further comprises a plurality of disc-like caps, cups or bearings, each size implant's collar having a central opening of the same size and shape so that each cup/cap/bridging bearing for that size implant can universally fit into its respective implant.

Other objects of the invention are achieved by providing an anatomic shaped orthopedic joint replacement implant wherein the head of the joint replacement device for a 1st MTP joint, the lesser MTP joints, and the elbow would have a ridge and groove, such as the ridge that manipulates the sagittal head called the “crista” on a 1st MTP, for added stability of each of these artificial joint replacement, and a corresponding “groove” on the phalangeal side of a 1st MTP total joint replacement, or on any of the above mentioned human joints that were to be constructed with a ridge to have corresponding groove to receive it.

Other objects of the invention are achieved by providing an anatomic shaped orthopedic joint replacement implant wherein parts of or all of the implants' head or stem is porous, or has a porous or lattice structure made via 3D printing or other manufacturing methods, to be implemented in the following joints: 1st MPJ, Lesser MPFs, Ankle, elbow, proximal metacarpophalangeal's, Trapezio-Metacarpal Thumb, and the proximal interphalangeal joints.

Other objects of the invention are achieved by providing an anatomic shaped orthopedic joint replacement implant wherein the osteotomy performed uses a 1st MPJMTP (metatarsal-phalangeal) total joint, or lesser MTP total joints that captures the natural declination angle of which the phalanx sits at rest relative to the metatarsal bone, which is between 14 and 19 degrees depending on the patent's persons anatomy.

Other objects of the invention are achieved by providing an anatomic shaped orthopedic joint replacement implant wherein the cap/bearing set of bearings to be used implement technology we refer to as “bridging bearings”, wherein such a bearing bridge the gap between one sized metatarsal implant and a different sized phalangeal implant, such as for the 1st Metatarsal-phalangeal Joint, Lesser MPJ's, but also to be used in total Ankle, elbow, proximal metacarpophalangeal's, Trapezio-Metacarpal Thumb, and the proximal interphalangeal joints to mix and match different sized implants together.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE FIGURES

FIG. 1 is a top rear perspective view of a three-dimensional anatomic first metatarsal-phalangeal joint replacement;

FIG. 2 is a top front perspective view thereof;

FIG. 3 is a right view thereof;

FIG. 4 is a left view thereof;

FIG. 5 is a top view thereof;

FIG. 6 is a bottom view thereof;

FIG. 7 is a back view thereof;

FIG. 8 is a front view thereof;

FIG. 9 is an exploded top front perspective view thereof;

FIG. 10 is an exploded bottom front perspective view thereof;

FIG. 11 is an exploded top front perspective view thereof;

FIG. 12 is an exploded bottom front perspective view thereof;

FIG. 13 is a cross sectional view thereof taken along line 13-13 in FIG. 9;

FIG. 14 is cross sectional view thereof taken along line 14-14 in FIG. 9.

FIG. 15 is a metatarsal implant left view thereof;

FIG. 16 is a metatarsal implant bottom view thereof;

FIG. 17 is a metatarsal implant rear view thereof;

FIG. 18 is a metatarsal implant top view thereof;

FIG. 19 is a metatarsal implant front view thereof;

FIG. 20 is a metatarsal implant front view thereof;

FIG. 21 is a proximal phalanx implant top view thereof;

FIG. 22 is a proximal phalanx implant top right view thereof;

FIG. 23 is a proximal phalanx implant top left view thereof;

FIG. 24 is a proximal phalanx implant rear view thereof;

FIG. 25 is a proximal phalanx implant front view thereof;

FIG. 26 is a proximal phalanx implant bottom view thereof;

FIG. 27 is a proximal phalanx implant bridging bearing front view thereof;

FIG. 28 is a proximal phalanx implant bridging bearing top view thereof;

FIG. 29 is a proximal phalanx implant bridging bearing top right view thereof;

FIG. 30 is a proximal phalanx implant bridging bearing left view thereof;

FIG. 31 is a proximal phalanx implant bridging bearing top left/front view thereof;

FIG. 32 is a proximal phalanx implant bridging bearing rear view thereof;

FIG. 33 is a fully assembled first metatarsal-phalangeal podiatric joint replacement top view thereof;

FIG. 34 is a fully assembled first metatarsal-phalangeal podiatric joint replacement right view thereof;

FIG. 35 is a fully assembled first metatarsal-phalangeal podiatric joint replacement Front view thereof;

FIG. 36 is a fully assembled first metatarsal-phalangeal podiatric joint replacement bottom view thereof;

FIGS. 37-58 are views of a view of a three-dimensional anatomic first metatarsal-phalangeal joint replacement system with a lattice structure.

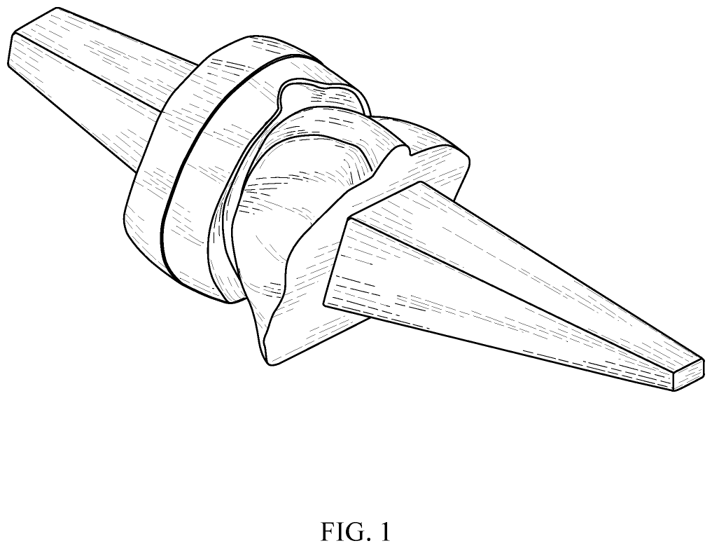

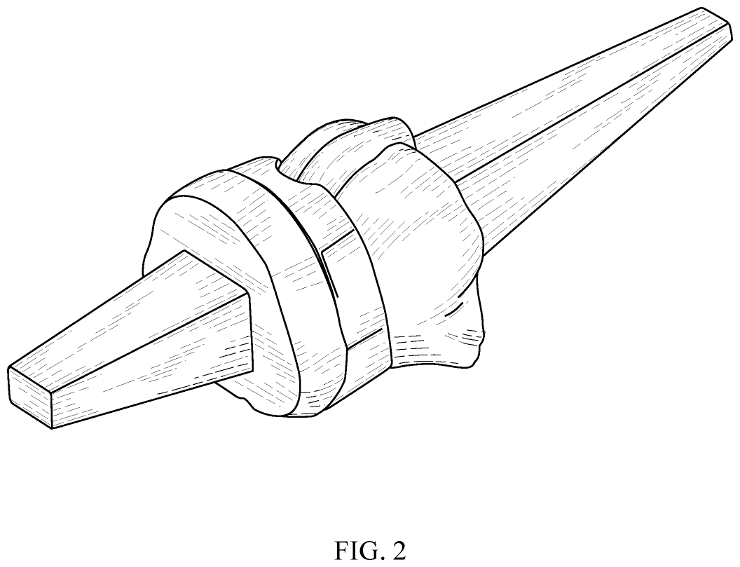

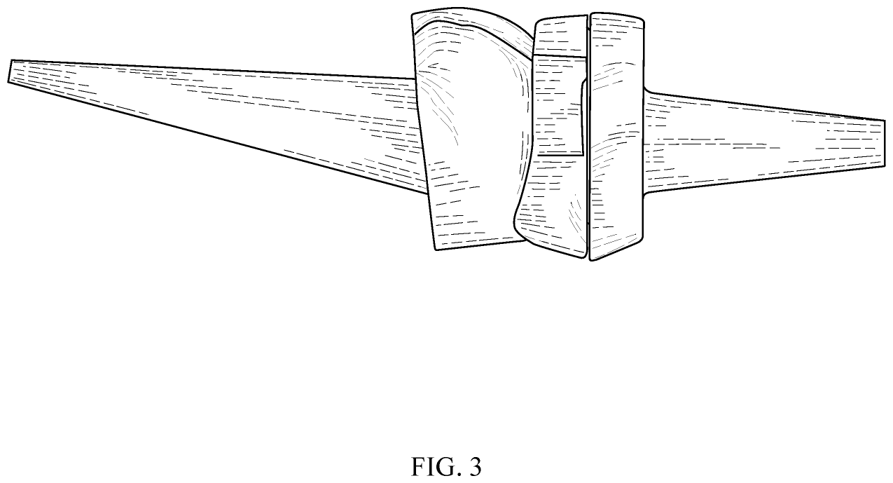

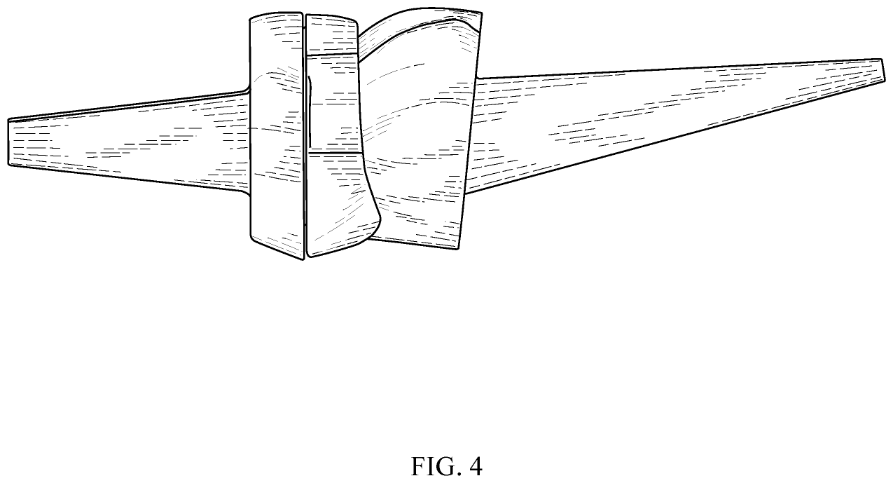

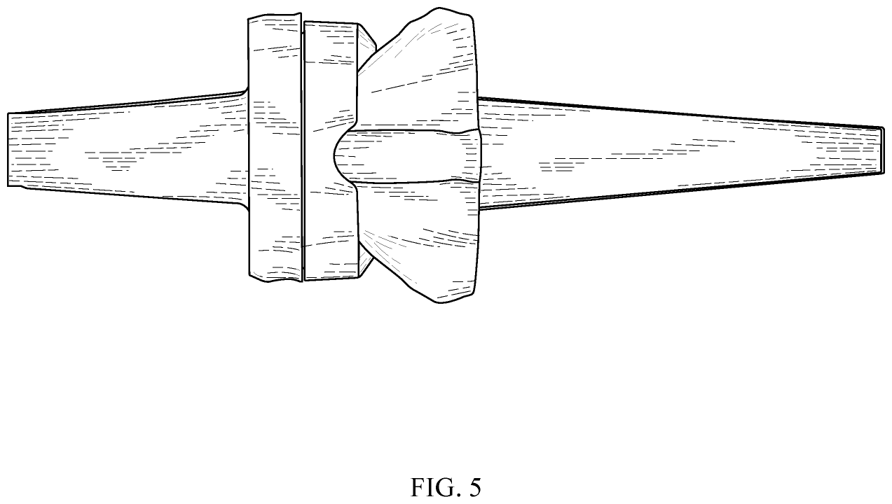

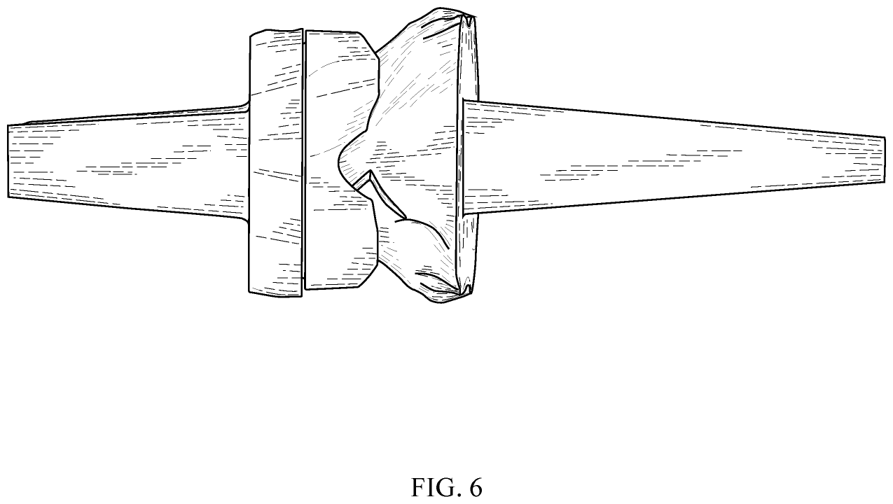

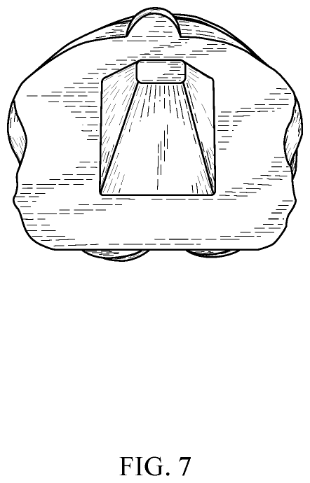

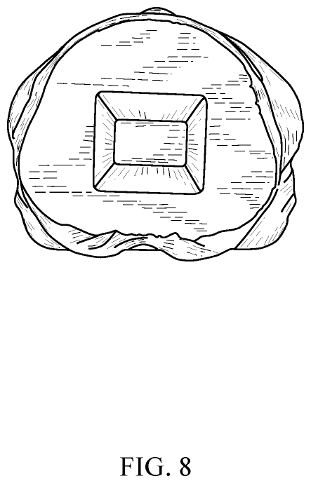

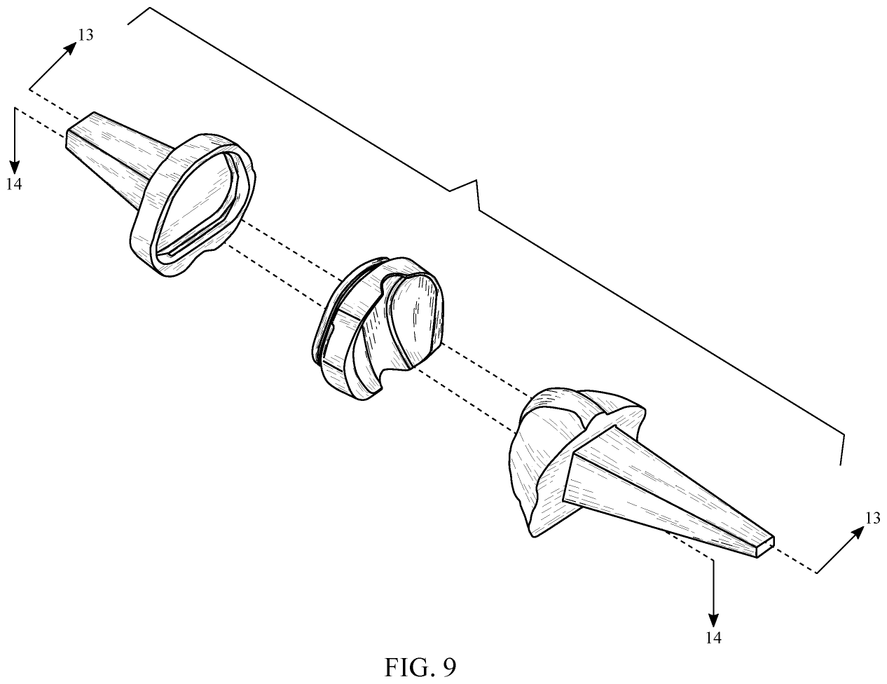

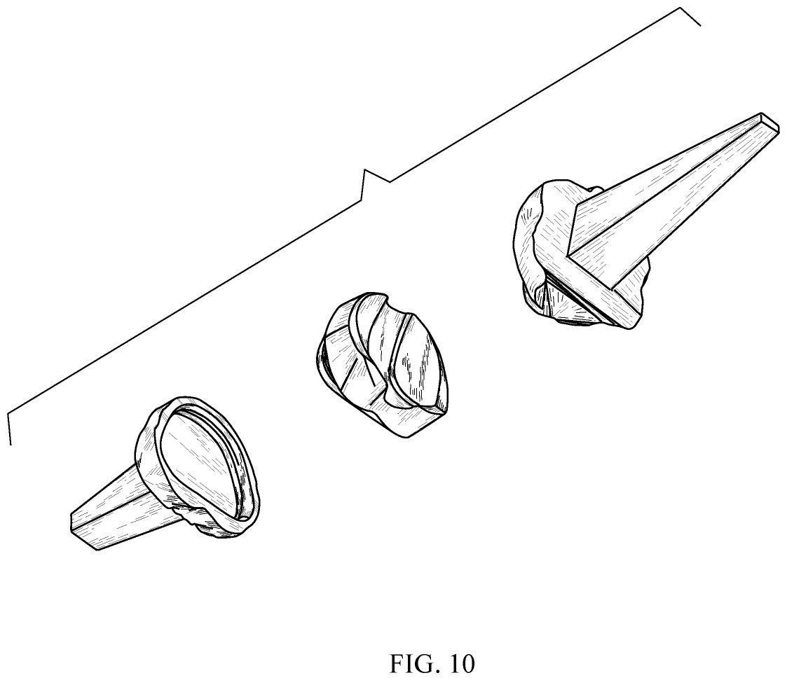

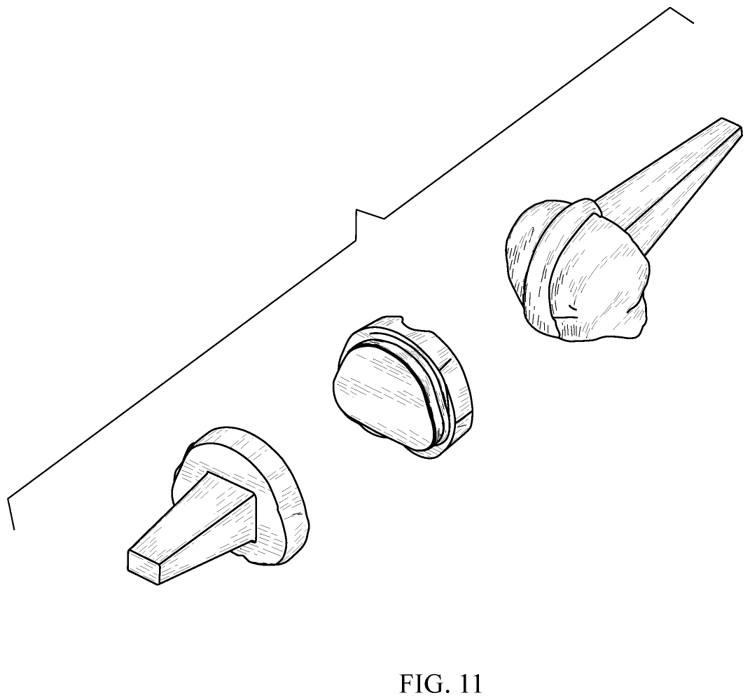

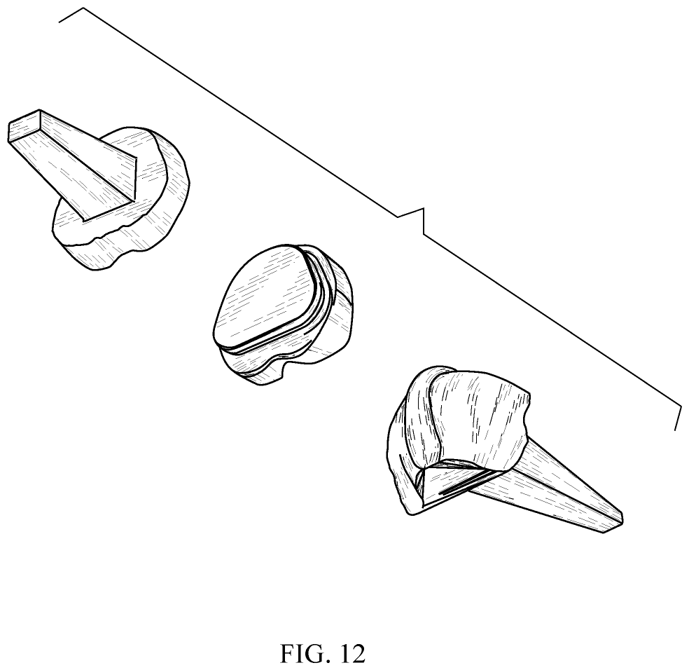

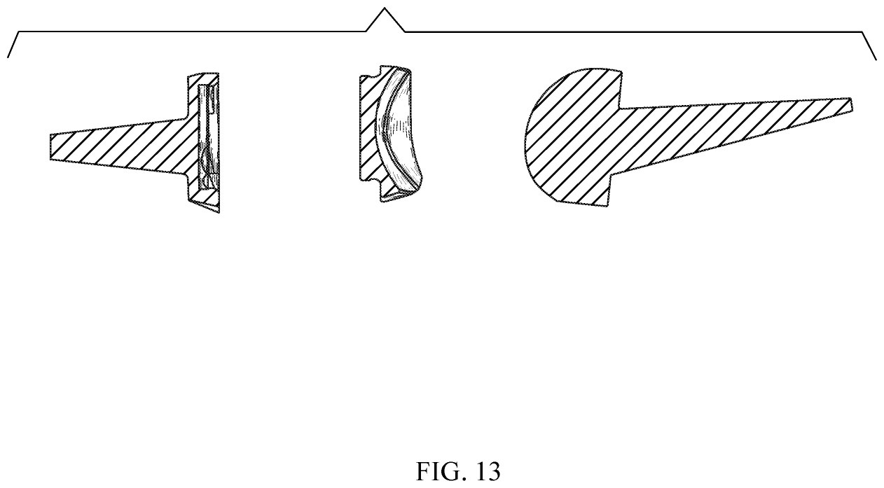

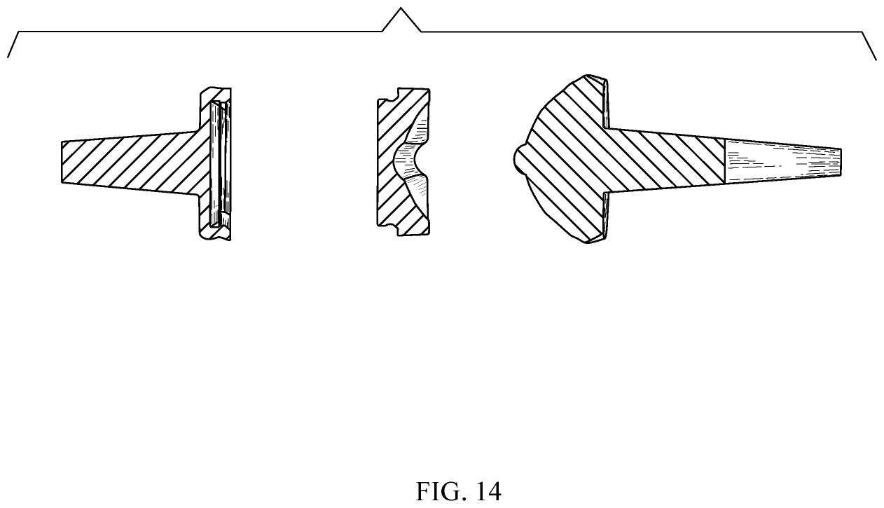

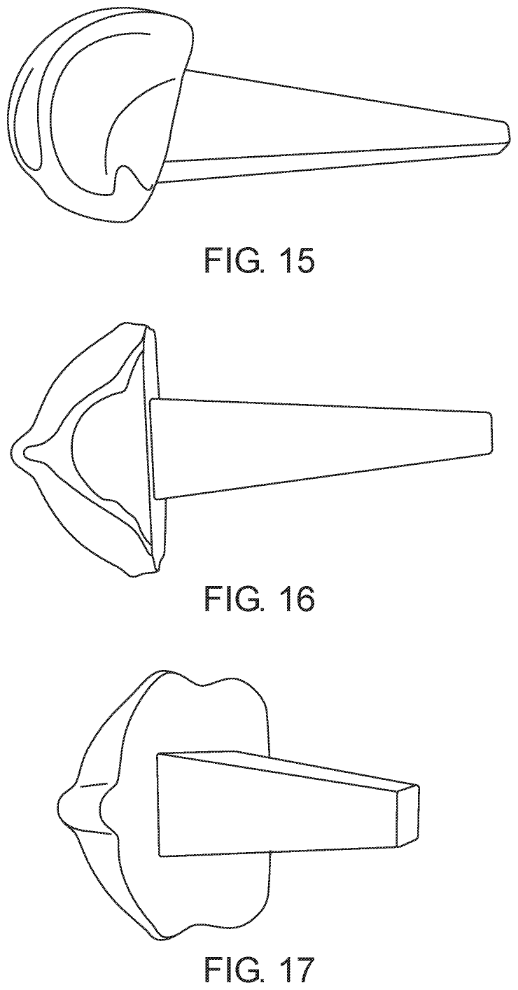

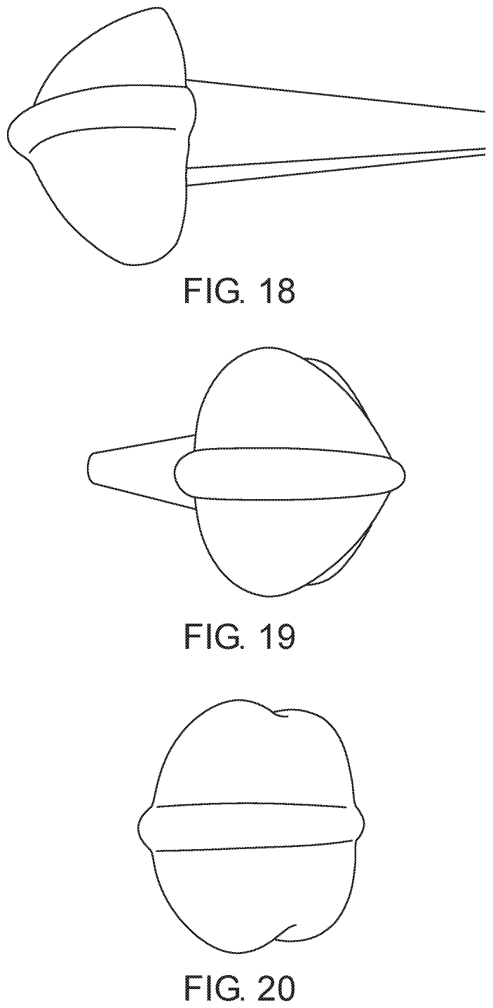

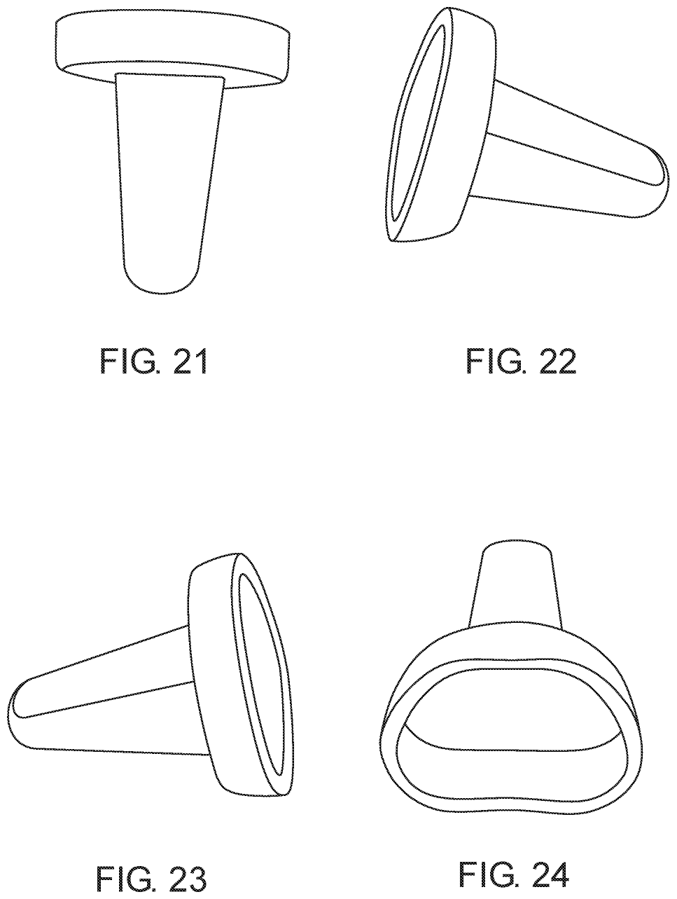

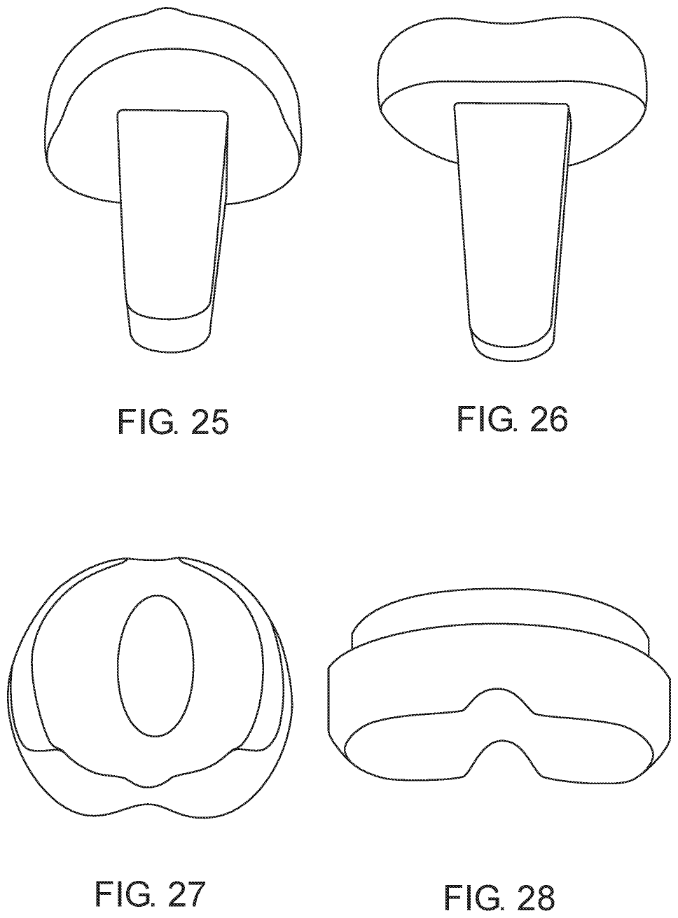

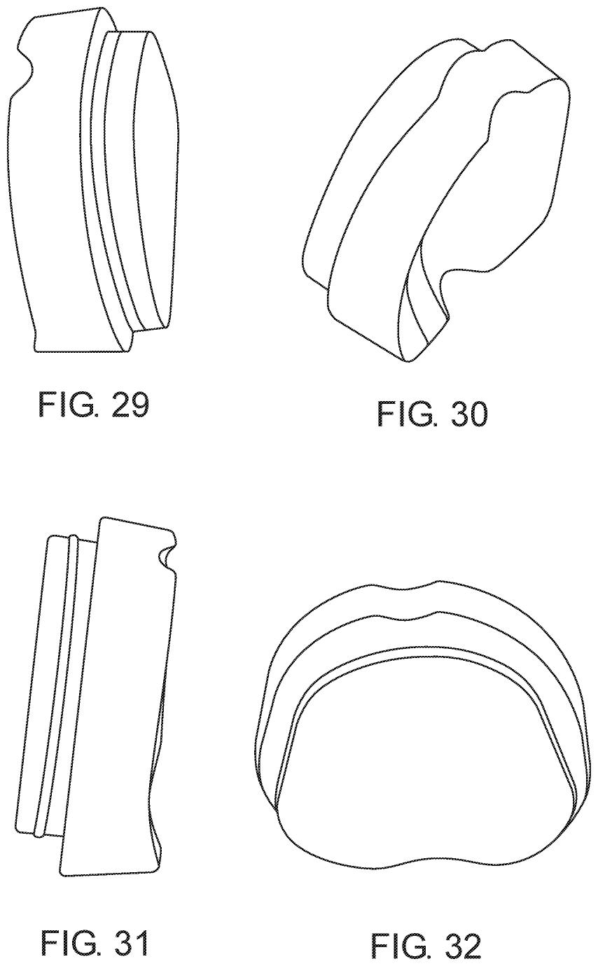

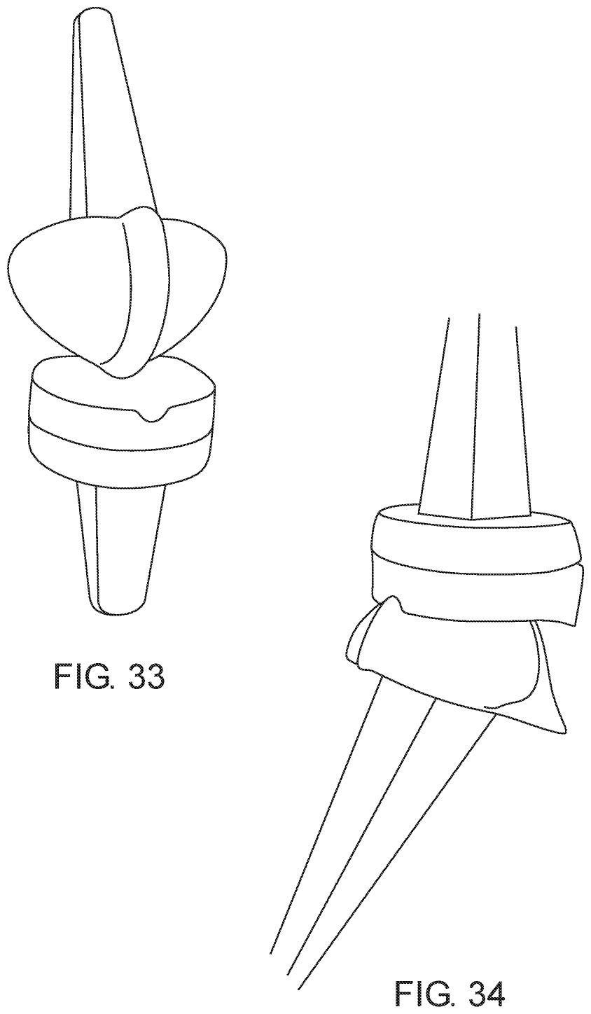

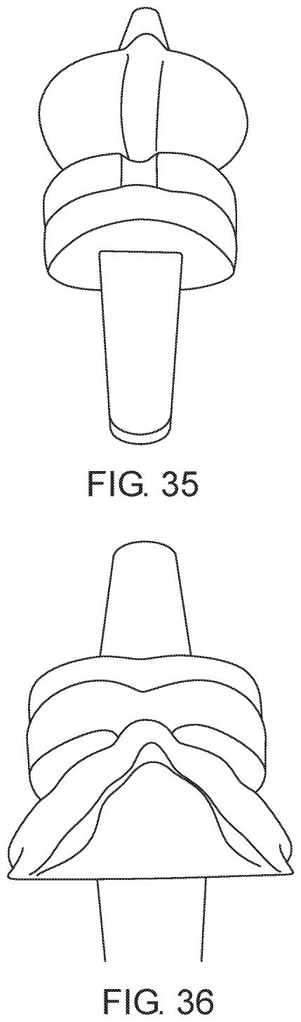

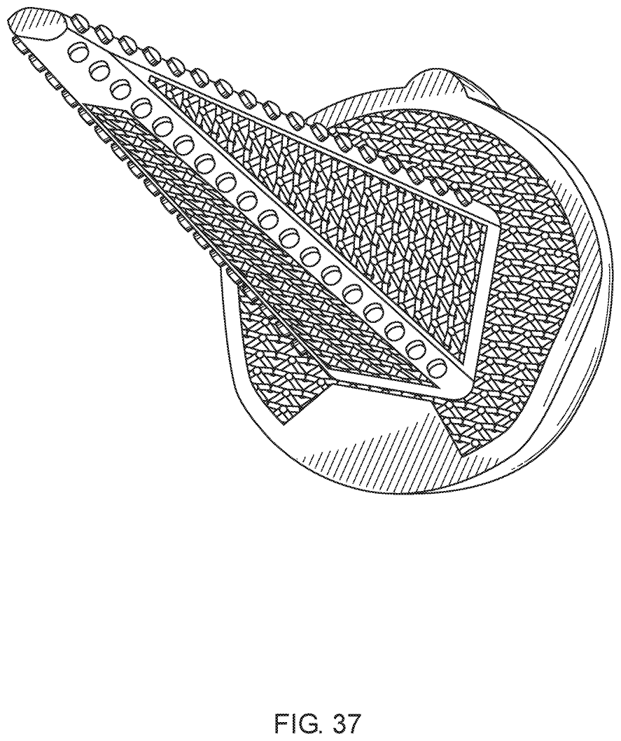

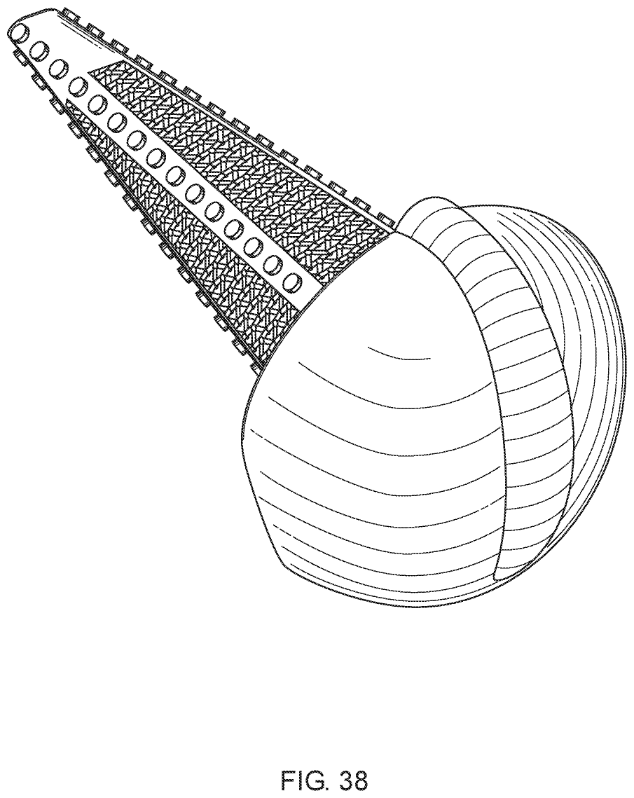

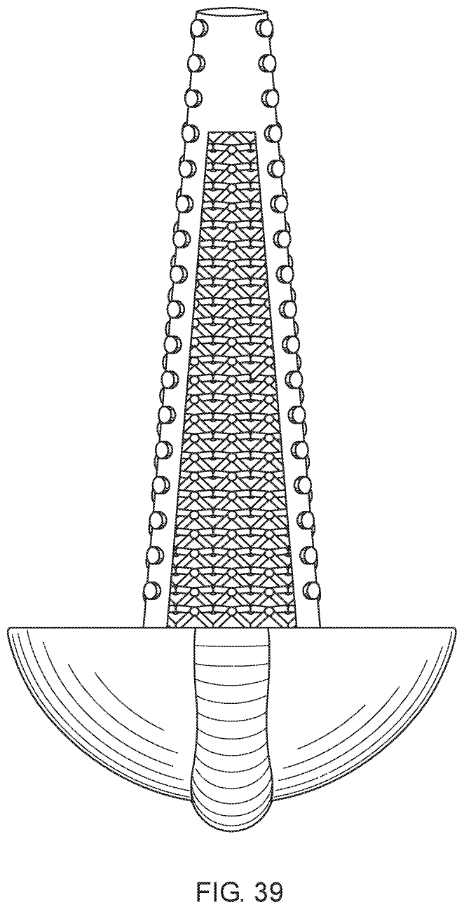

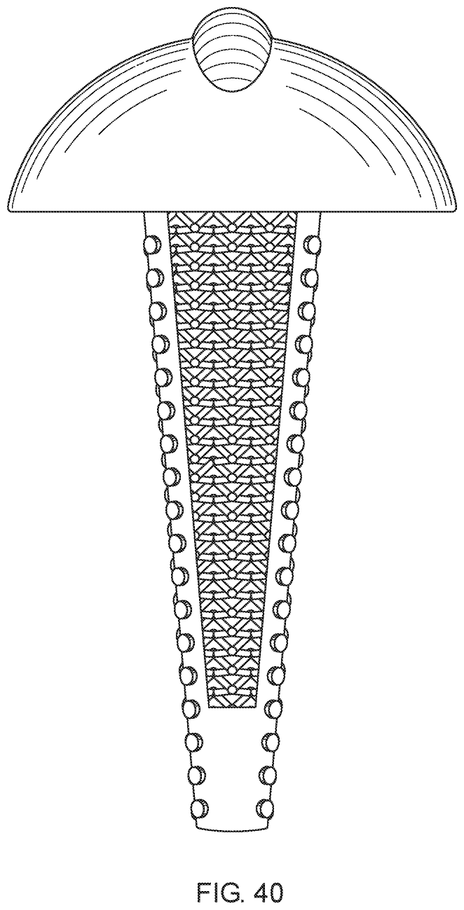

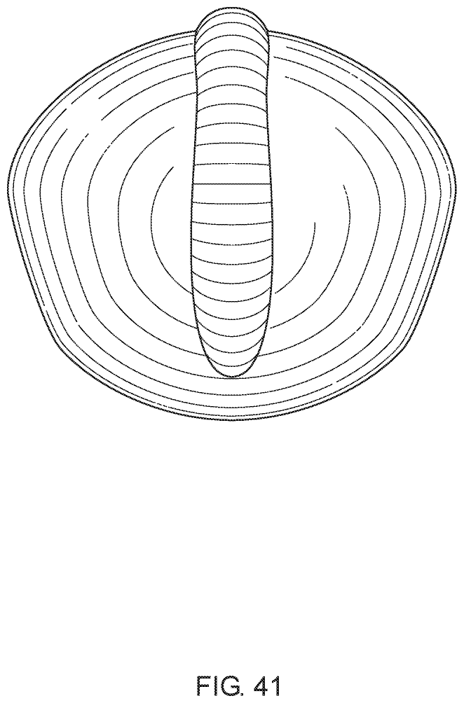

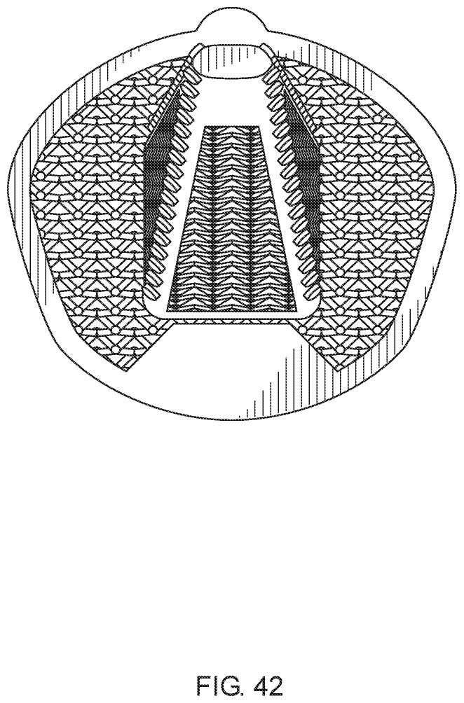

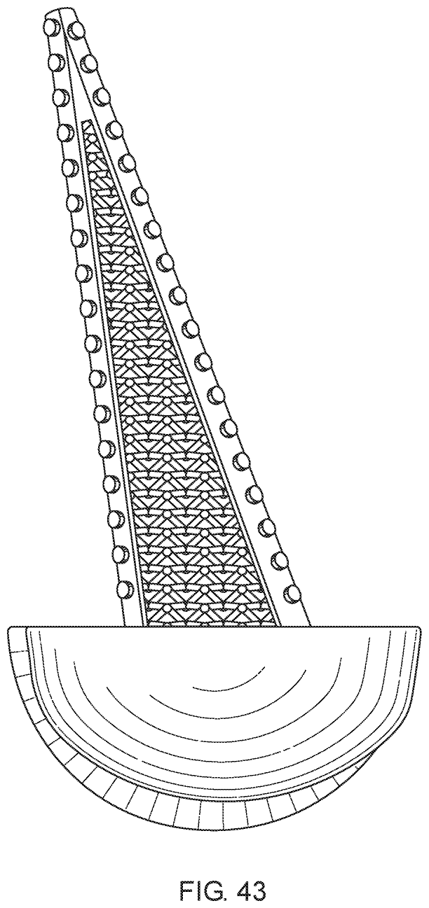

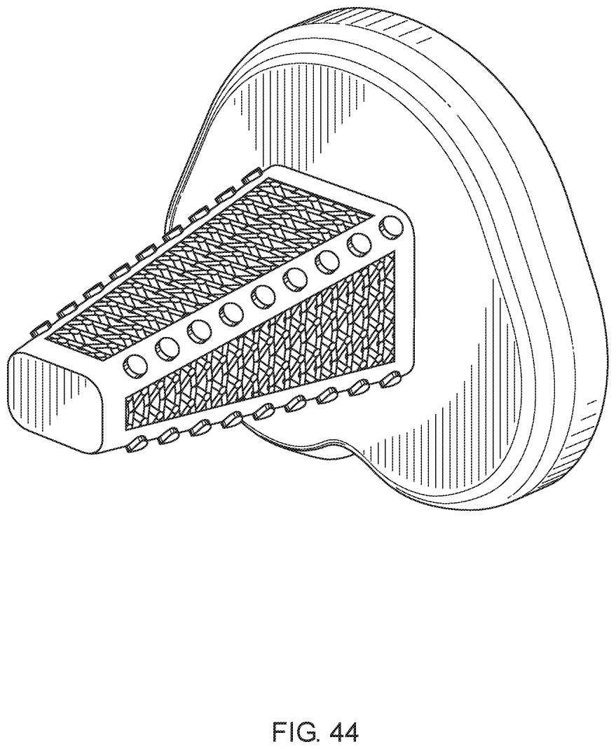

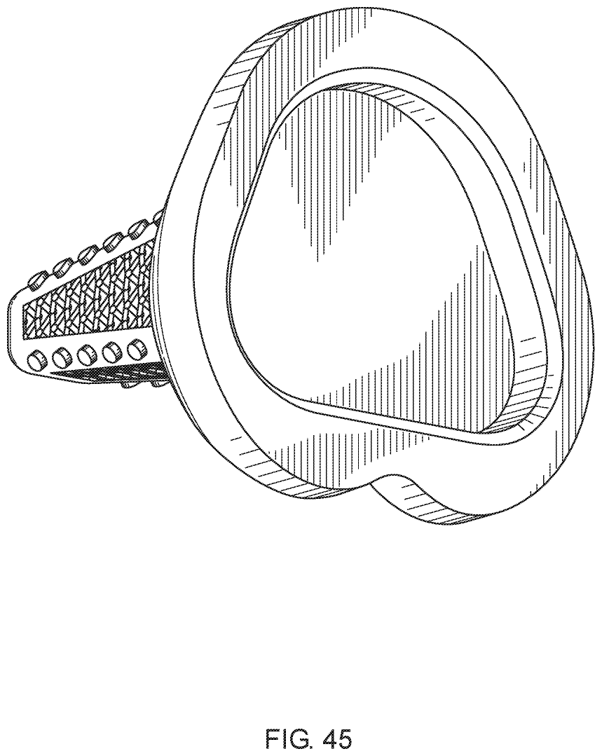

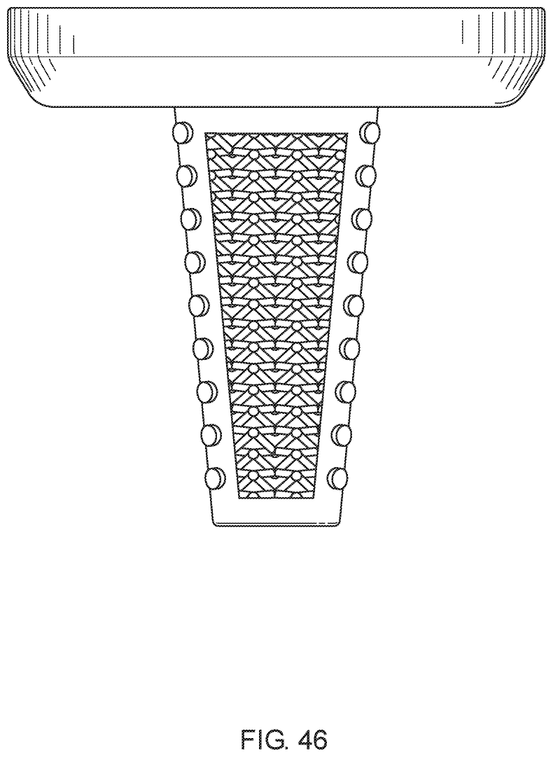

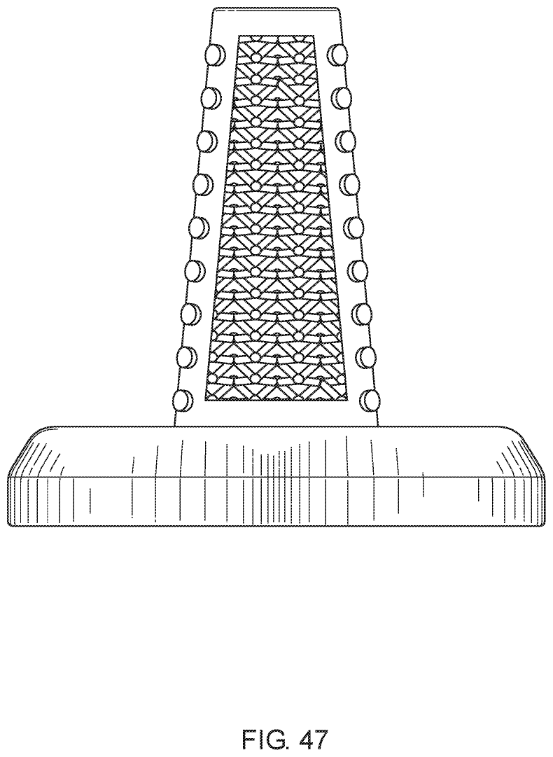

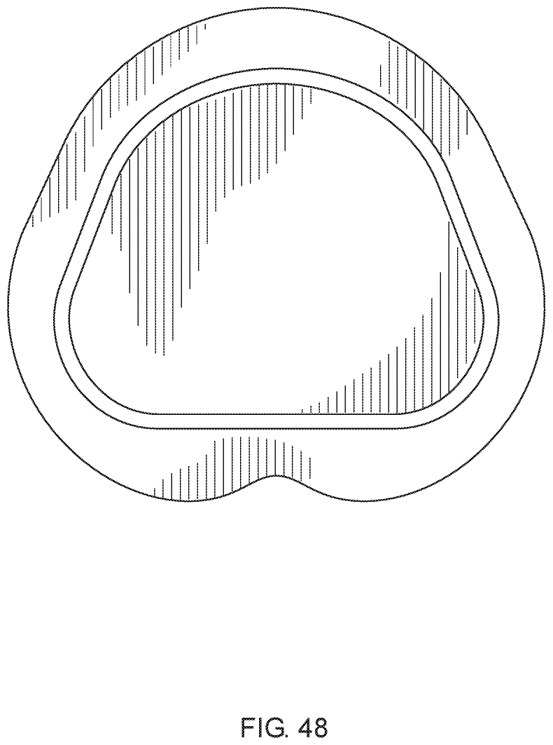

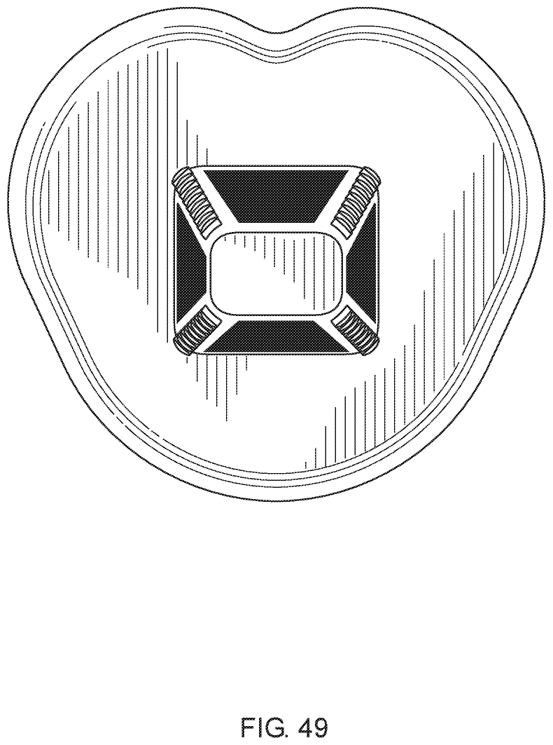

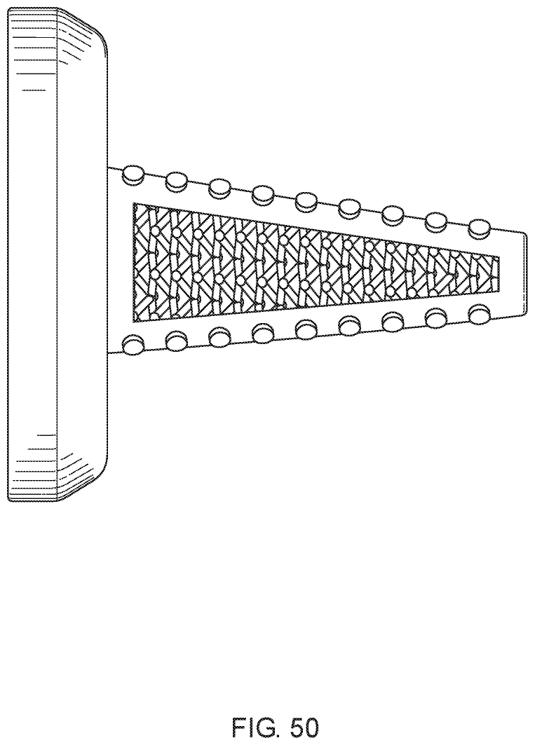

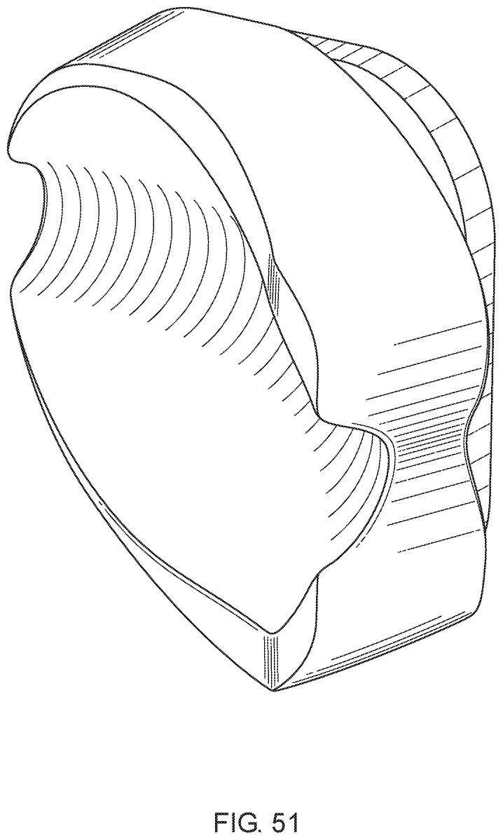

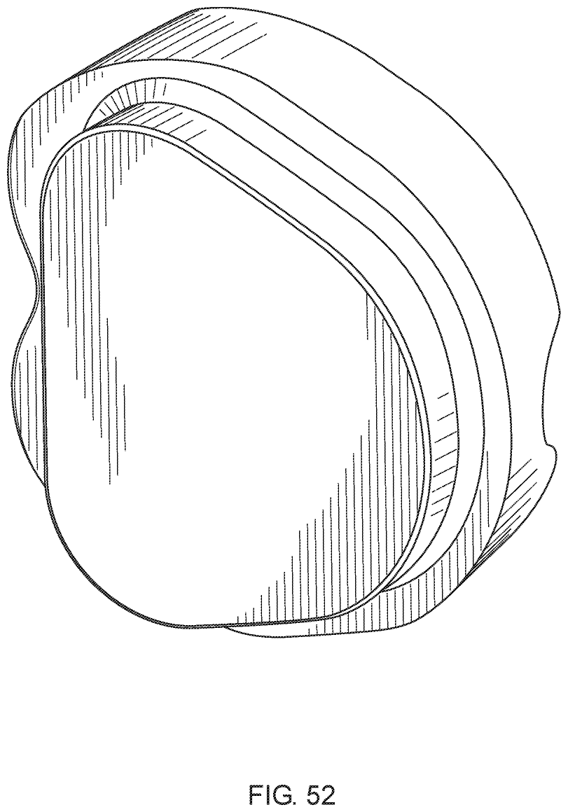

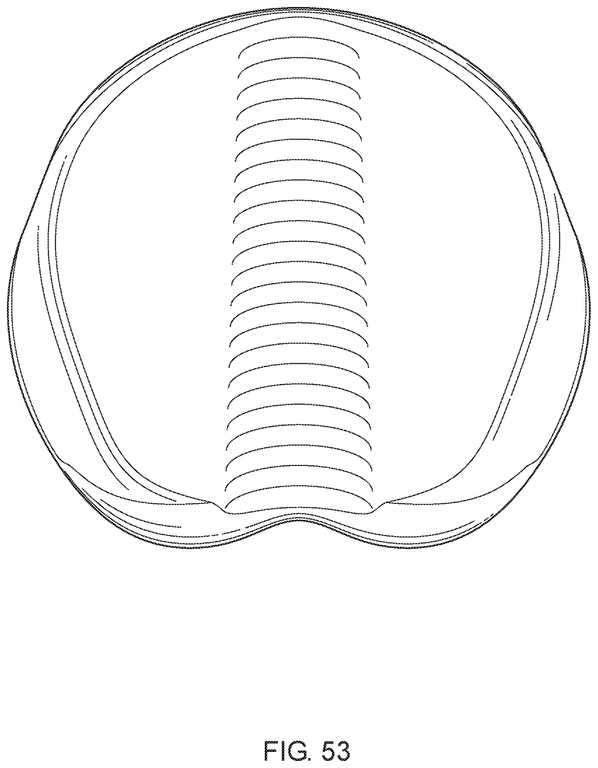

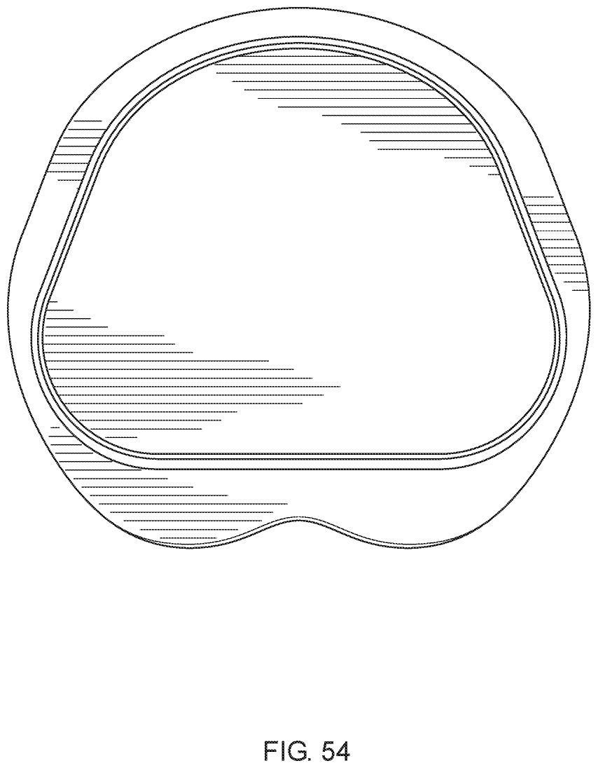

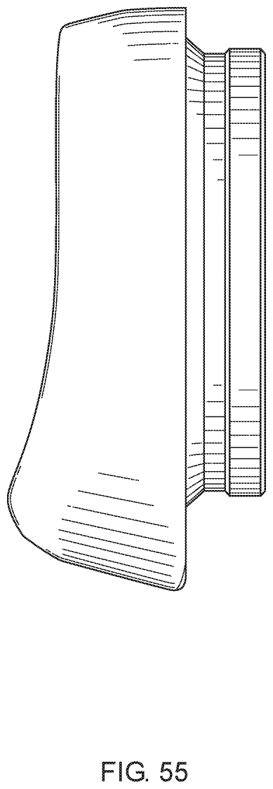

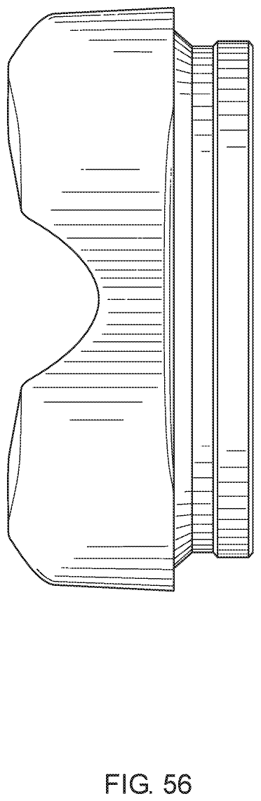

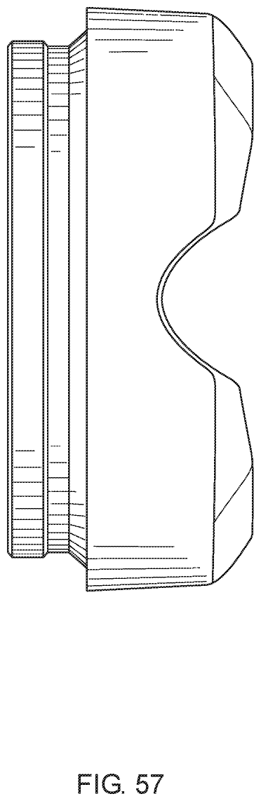

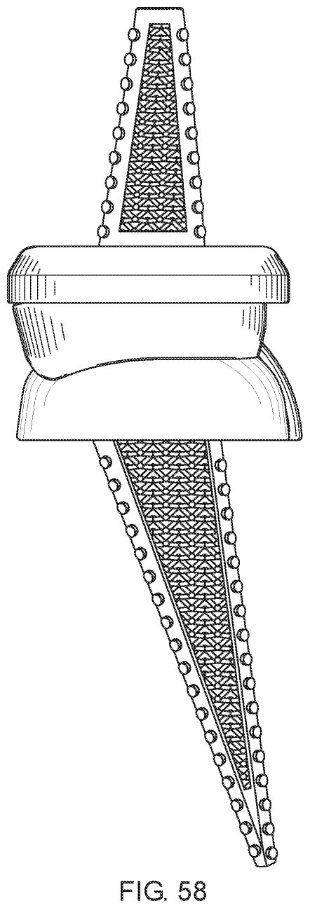

### DETAILED DESCRIPTION OF THE INVENTION

In the following description, numerous details are set forth for the purpose of explanation. However, one of ordinary skill in the art will realize that the invention may be practiced without the use of these specific details.

This invention is intended for systems and methods of manufacturing and use of improved implants for hip, knee and shoulder and elbow joint replacements, as well as podiatric joints including the ankle, and hand/wrist joint replacements including finger joints, utilizing the method of laser scanning or any other type of 3D imaging of select human bones for each partial or total joint replacement to be modeled after.

In certain embodiments, the method is directed to the manufacturing (of knee and elbow joints, as well as podiatric joints including the ankle, 1^{st} MPJ, lesser MPJ's, and hand/wrist joint replacements including finger joints). of joint replacements that may come from using the present method of manufacture.

In certain embodiments, the methods and systems include creating implants having a stem structure that is round shaped, square shaped, as well as the hybrid versions that would be a result of any kind of merging of those 2 shapes.

In certain embodiments, the methods and systems include creating implants with any or all of the above-mentioned features, and to include but not limited to having caps that serve as bridging bearings. A bridging bearing is defined as a poly or plastic like component that bridges the gap between one implant and the other. It is often the middle or “in between” component of the total joint replacement that is the buffer between the main 2 metal components. This can be used in joint replacements as well as total joint replacements. For example, the implants in a set of 5 total sizes for the metatarsal-phalangeal joint replacement would be named M1 for the smallest and M5 for the largest metatarsal pieces, with P1 being the smallest size and P5 being the largest size for the phalangeal side. A bridging bearing would serve the purpose of letting the surgeon be able to use a M1 implant with a P2 implant through the use of using a P2-1 bridging bearing (the 2 standing for the size phalanx the bearing corresponds to snapping into and the -1 part corresponding to the size metatarsal implant that the bearings face shape fits perfectly over. It works the same way for matching a P2 with a M3 bearing with the use of a P2-3 bearing/cap. In this example, the size P2-4 phalanx implants can use bridging bearings to be compatible with 3 different sized metatarsal implants, while P1 and P5 can only do their corresponding size as well as one additional (One size larger for the P1 implant and one size smaller for the P5 implant) meaning that in this set there would be a total of 13 bridging bearings for 5 different sized implants. The main function of the bridging bearing/bridging caps are for patients that may have a joint where one of the bones' inner canal isn't proportional to the corresponding bones' canal, thus using one size implant on one side with a different sized implant on the other side of the joint. This bridging bearing technique may be used in combination with some or all of the above-mentioned features, not only for total joint replacements but for partial as well, wherein only one bone end of a joint is resurfaced and one implant is used.

In certain embodiments, the methods and systems include creating an implant where the implant's head is or is not permanently affixed to the stem for partial/total joint replacements, including but not limited to having an implant head that may swivel on the stem.

In certain embodiments, the methods and systems include creating implants wherein the head of an implant (for any pieces of a total joint replacement system) includes one or multiple long shaped protrusions coming out of the face of the head (such as the crista on a natural 1^{st} Metatarsal head), for the purpose of guiding the opposing piece specifically in the longitudinal direction to prevent subluxation of the artificial joint.

In certain embodiments, the methods and systems include creating total joint replacement wherein the adjacent implant's head has a corresponding groove to fit the long-shaped protrusion that acts as the stabilizing agent.

In certain embodiments, the methods and systems include creating implants wherein parts of or all of the entire stem is porous.

In certain embodiments, the methods and systems include creating implants wherein part of an implants head (including but not limited to where the back side of the implant head meets the stem, on that plane of the implants head, as well as within the head itself being porous) is porous.

In certain embodiments, the methods and systems include creating implants wherein the largest 2 components of the total joint (by volume) of the implant system are made of a solid piece of alloy, metal, composite or other material.

In certain embodiments, the methods and systems include creating implants wherein the head of an implant is positioned at a declination angle in relation to the implants stem, to match the natural declination angle of how the pathological bones naturally sit when at rest.

A detailed step by step description of an exemplary method is included below with examples.

In certain embodiments, the method involves providing a surgeon within the field of that joint will then be brought in to consult a designer of the joint in all steps of design and possibly some or all steps of manufacture, the first of which will be to determine the locations of where the cut should be made on the one bone for a partial joint replacement (and each bone for a total joint replacement), and making indicators/markings for the designer to use for later reference in order to fabricate the implant to conform to the dimensions of the cortical bone in the region of the osteotomy, after which a stem/anchor is designed and attached or made to fit on to the collar or head of the implant/implants, along with any needed caps/bearings/bridging bearings or heads needed.

In certain embodiments, the implantable joint replacement is to be used for insertion into the medullary canal of a bone upon which an osteotomy has been performed, of which the implant's stem adapted to extend into the medullary canal of a patient's bone, wherein the implant/implants are for joints in body locations selected from the group consisting of elbow, wrist, hand, ankle, foot, hip, knee and shoulder.

In certain embodiments, the method involves the use of any structure manipulation methods including but not limited to cast molding, laser scanning, x-ray, magnetic resonance imaging, inverted 3D panoramic photography by taking pictures of the bone from multiple different angles and putting them in a computer program to construct a 3D image of the bone, and any other ways one could construct a 3D image or model of manipulating a human bone, etc.

In certain embodiments, a number of human bones (of each desired implant to be designed) are acquired and used as the model of which to design the implant/implants after. The number of bones acquired will be at the discretion of the designer in relation to how many sizes of each implant they want to make. For example, if the designer plans to manufacture 5 sizes of a total joint replacement implant for the 1^{st} MPJ (first metatarsal-phalangeal joint, the smallest size being size #1, and the largest being size #5 for both metatarsal and phalangeal bones), that designer may have to acquire 5 metatarsal bones as well as 5 phalangeal bones of equal incremental increase in size.

In certain embodiments, if the designer wanted to, they could also choose to acquire only the smallest size metatarsal and phalangeal bones, as well as only the largest size metatarsal and phalangeal bones, measure the outer dimensions of each and use the average of the largest 2 metatarsal bones (sizes metatarsal #5 and phalangeal #5) to come up with the middle sized metatarsal bone which would be size #3. Then they could use the average of size #3 and size #5 to create size #4, as well as using the average of size #'s 1 and 3 to determine size #2. The metatarsal and phalangeal bones for each modeled size would most likely need to come from the same human body for reasons of proportion.

In certain embodiments, the designer will create a 3D file and will alter the file so that the part of the bone (where the joint resurfacing most commonly starts) that is cut off in surgery will be segregated and saved into a new file to act as the head of the new implants in the set (the head being defined as the perpendicular part of each implant that meets or connects to the stem, or the horizontal part of a “T” with the vertical part being the stem). For example, for a first metatarsal-phalangeal total joint replacement, the head of the metatarsal would be cut off where the consulting surgeon indicated, as well as the base of the phalanx as was indicated. The metatarsal head from the modeled human bone that was segregated would act as the model for the metatarsal implant's head, and the base of the phalanx bone that was segregated would serve as the model for the designing of the phalangeal implant's collar (the collar is defined as the agent meant for housing the cupping agent/bearing/cap of which both make up the base of the phalangeal implant) that would “cup” the metatarsal head. The head portion of an implant would have dimensions on the back side of the head that would approximate the outer diameter of the bone at the site of the modeled osteotomy. The base part of the phalangeal implant (not the bone) is defined as being comprised of both a collar and a bearing/cap/cup.

In certain embodiments, the collar has an outer circumference that approximates the outer circumference of one modeled bone, along with the head approximating the size and shape of the other modeled bone, and for total joint replacements having the implant with the collar being rotatable about the longitudinal axis of the implant with the head.

In certain embodiments, the implant that houses the cupping agent/bearing/cap is defined as the implant with a collar that rotates along the longitudinal axis of the implant with the head. For example, in a first metatarsal-phalangeal total joint replacement, the implant with the semi-spherical shaped head attached to the stem would be used to resurface the head of the metatarsal bone, and the implant with the cupping part would be defined as the implant with the collar that resurfaces the base of the phalangeal bone. The implant with the cupping agent that rotates along the head is traditionally implanted into the joint osteotomy in the joint that defined as the lower part of the digit/limb, with the implant having the semi-spherical shaped head traditionally being implanted into the joint osteotomy that is defined as the upper part of the limb/digit for fingers/toes/elbow, knee and ankle

In certain embodiments, the implant designer (including but not limited to a CAD artist, program or algorithm as well) may slightly alter the modeled bone's cortical surface or surfaces to erase any imperfections or deviations from the natural obvious contours of that bone, for the purpose of making perfectly smooth surfaces for the collars/heads of the implants in the set.

In certain embodiments, the implant designer may or may not choose the most desired half of the bone's head of which to model the implant's head after. For example, the designer may choose to cut the head in half in the program, duplicate the most desired half in a mirrored image and attach it to the original preferred half. The purpose of this is to have the most desired half of the modeled bone's head serve to make a perfectly symmetrical head/base for each implant. This part of the method may be used during a number of circumstances, including but not limited to when one half of a bone's head is not symmetrical to the other half

In certain embodiments, stems or anchors will then be designed of varying sizes, and will most likely increase incrementally and proportionally to the size of each implant. The stems will most often be tapered in the longitudinal axis from wide up at the head and decreasing in volume as the end of the stem is approached.

In certain embodiments, the stem defines a longitudinal axis and is round, square or a marriage of the two in cross section; the post is of longitudinal axis and is semi-circular in cross section. Stem and anchor designs will most likely vary at the discretion of the designer and the consulting surgeon.

In certain embodiments, the determining the dimensions of each implants stem include but are not limited to: The consulting surgeon modeling the implant's stem (for each size implant) off of the medullary canal dimensions taken from the bones that were acquired to be used as the models. For example, if the designer plans to manufacture 5 sizes of a total joint replacement implant for the first metatarsal-phalangeal joint (the smallest size being size #1, and the largest being size #5 for both metatarsal and phalangeal bones), that designer may have to acquire 5 metatarsal bones as well as 5 phalangeal bones of equal incremental increase in size. If the designer wanted to, they could also choose to acquire only the smallest size metatarsal and phalangeal bones, as well as only the largest size metatarsal & phalangeal bones, measure the dimensions of the medullary canals for all 4, and use the dimensions of the largest and smallest sized metatarsal medullary canals to determine the dimensions of the middle sized implant's stem/anchor which would be size #3. The largest bone (for both metatarsal and phalangeal) would serve as the model for size #5 in an implant set of 1-5, and the smallest size bone for both would serve as the model for size #1 in an implant set of 1-5. Then the implant designer could use the average of the determined stem/anchor size #3 and size #5 to create size #4, as well as using the average of stem/anchor size #'s 1 and 3 to determine size #2.

In certain embodiments, the designed heads of the implants will then be affixed to their respective stems/anchors (for example metatarsal head 5 would correspond to metatarsal stem 5, and phalanx head 5 would correspond to phalanx stem 5, etc.). These stems/anchors will most likely be the main feature that connects the implant's head flush to the remaining part of the bone not removed from the patient during surgery, thus resurfacing the joint/joints. Multiple sizes should be made to accommodate different size bones or bone structures in different patients. Once the partial or total joint replacement design is finished, the 3D files or molds/3D models can then be used to manufacture the new joint replacements via methods to include but not limited to 3D printing, cast molding, direct metal laser sintering, machining processes such as CNC, etc. The implants are to be composed out of materials including but not limited to titanium alloys, 316L stainless steel, cobalt chromium, tantalum, Polyethylene, UHM Polyethylene, ISO Polycarbonate, polyurethane, and any other FDA approved materials that the manufacturing company chooses.

In certain embodiments, the implant includes a collar having first and second opposing sides, a stem that projects from one side of the collar and which extends into the bone at the site of the osteotomy, and a cap/bearing that is connected to the post to complete the formation of the implant's head.

In certain embodiments, the method further comprises comprising the step of providing “a plurality of disc-like caps”, cups or bearings, each size implant's collar having a central opening of the same size and shape so that each cup/cap/bridging bearing for that size implant can universally fit into its respective implant. For example, a bridging bearing is defined as a type of cup/cap that bridges the gap between one implant and the other. This can be used in joint replacements as well as total joint replacements. For example, say the implants in a set of 5 total sizes for the metatarsal-phalangeal joint replacement would be named M1 for the smallest and M5 for the largest metatarsal sizes, with P1 being the smallest size and P5 being the largest size for the phalangeal side. A bridging bearing would serve the purpose of letting the surgeon be able to use a M1 implant with a P2 implant through the use of using a P2-1 bridging bearing (the 2 standing for the size phalanx the bearing corresponds to snapping into and the -1 part corresponding to the size metatarsal implant that the bearings face shape fits perfectly over. It works the same way for matching a P2 with a M3 bearing with the use of a P2-3 bearing/cap. In this example, the size P2-4 phalanx implants can use bridging bearings to be compatible with 3 different sized metatarsal implants, while P1 and P5 can only do their corresponding size as well as one additional (One size larger for the P1 implant and one size smaller for the P5 implant) meaning that in this set there would be a total of 13 bridging bearings for 5 different sized implants. The main function of the bridging bearing/bridging caps are for patients that may have a joint where one of the bones' inner canal isn't proportional to the corresponding bones' canal, thus using one size implant on one side with a different sized implant on the other side of the joint. This bridging bearing technique may be used in combination with some or all of the above-mentioned features, not only for total joint replacements but for partial as well, wherein only one bone end of a joint is resurfaced and one implant is used.

In certain embodiments, the bridging bearings caps/cups are designed by manipulating the shape of the head of each implant in a given set. For example, for implants in a set of 5 total sizes for the metatarsal-phalangeal total joint replacement, the metatarsal heads of each size would be press fitted into a mold or have a 3D image taken of the head's forward facing surface. Once the image or mold has been achieved, several caps/cups would be made for each phalangeal implant's collar (the collar being defined as the part of the implant where the bearing/cap/cup is affixed to or where it snaps into). If a phalangeal implant had 3 cups/caps/bearings per implant, then all 3 of those cups/caps/bearings would have a similar inner non-circular shape on the back side of them that allows for them to be universally switched out for each other on that one phalangeal implant, allowing for the side with the non-circular shape to snap into the collar. However, all 3 of these mentioned cups/caps/bearings would correspond to 3 different metatarsal head implants (as their goal is to perfectly cup one of the other 3 implant's head's). The same goes for implants who's designs will have more or less than 3 cups/bearings/caps per implant with a collar. How many of these cups/caps/bearings per implant will be left up to the discretion of the implant's designer.

In certain embodiments, all of the elements listed above are to be assembled into complete sets and distributed to hospitals/orthopedic surgery centers as needed for patients that need the joint replacements.

### How The Invention Works:

The invention works in that it will be used to create joint replacements/total joint replacements that are an anatomic in nature (modeling human bones). The set created for each joint replacement will have several different size implants (for each joint in the body that the manufacturer makes it for.

This example holds the same application not only for metatarsal-phalangeal joint replacements but also other joints such as knee, elbow, ankle, wrist, and finger joints. This method is for use in replacing a total joint, or a partial joint by removing the end of the osteotomy of the one or 2 bones, and replacing one or both with the new partial joint or total joint implant/implants. The series of steps on the manufacture and surgical methods are included below:

### How To Make The Invention:

In one embodiment, a method to manufacture a joint replacement for the first metatarsal-phalangeal joint is as follows:
1. For which ever joint replacement is being manufactured, one would first acquire a human bone or bones of that joint in which to model the implant after. The human bones ideally would not have any joint diseases.
2. Acquire and produce a 3D image/model of the desired human bone. Modeling methods include but are not limited to any kind of laser scanning, digital or x-ray scanning, infrared methods, as well as taking a mold/casting of the bone and ALL other exact methods of replication including but not limited to magnetic resonance imaging, inverted 3D panorama photography by taking pictures of the bone from multiple different angles and putting them in a computer program to construct a 3D image of the bone, etc.
3. Make a 3D computer file of the whole bone in solid works or any other desired professional 3D illustrating program/algorithm that would most likely include but not be limited to STL, STEP, IGES or other 3D compatible formats.
4. The implant creator/program/algorithm may slightly alter the bones surface to erase any imperfections to make smooth surfaces for the heads of the implants in the set.
5. For joint replacements and total joint replacements, the designer will take the 3D file and alter it so that the part of the bone (where the joint resurfacing happens) that is cut off in surgery will be saved into a new file to act as the heads and bases/collars of the new implants in the set.
6. Stems or anchors will then be designed of varying sizes, and will most likely increase incrementally and proportionally to the size of each implant. Stem and anchor designs will most likely vary. The heads/collars of the implants will then be affixed to their respective stems/anchors (or whatever device someone chooses to patent for that particular part of the body). These stems/anchors will most likely be the main feature that connects the implant's head flush to the remaining part of the bone not removed from the patient during surgery, thus resurfacing the joint/joints. Multiple sizes should be made to accommodate different size bones or bone structures in different patients. Once the partial or total joint replacement design is finished, the 3D files can then be used to manufacture the new joint replacements via methods to include but not limited to 3D printing, cast molding, direct metal laser sintering, machining processes such as CNC, etc. How many sizes and variations will be left up to the company that produces the implant. The implants are to be composed out of materials including but not limited to titanium alloys, 316L stainless steel, cobalt chromium, tantalum, Polyethylene, UHM Polyethylene, ISO Polycarbonate, polyurethane, and any other materials that the manufacturing company chooses.

### How To Use The Invention:

The present invention is intended for use by surgical/orthopedic companies for the purpose of manufacturing and/or selling the new and better implants that will come from it. With this new method, these companies and entities like it will have the tools to develop many new and improved implants to help as many people needing joint replacements as possible. The proposed method outlined in this application includes but is not limited to a surgical method such as the one that is included below. It is a surgical method for the first metatarsal-phalangeal joint that will serve as an example of how one would use the inventions that come from this method. The method itself is however limited to the 1st MTP joint mentioned above.

1. The surgeon uses standard aseptic surgical technique for bunionectomy dissection.
1. A vertical cut is made to take off an average of 6-8 mm of bone material from the base of the proximal phalanx.
2. A 75-degree angled cut is made (off the longitudinal axis of the first metatarsal bone) at the head of the 1^{st} metatarsal starting at the subchondral bone level, to remove the arthritic first metatarsal head. Approximately 7-9 mm of the arthritic metatarsal head is removed, the wider portion being from the dorsal aspect at the previously mentioned 75-degree angle).
3. After the surgeon makes the 75-degree angle cut, the sizer instruments are used to determine which size implant in the set should be used on the patient. This is done by taking the instrument labeled “sizer” in the tray and placing them against each re-surfaced bone. For example, if the re-surfaced metatarsal head best overlaps the “M5” marking on the sizer instrument labeled “M”, that will indicate that the size “M5” implant will be the best fit for that patient. The same goes for the base of the Phalanx. If the sizer instruments indicate that the patient best matches to a “M2” implant for the metatarsal side and a “P1” implant for their phalangeal side, the surgeon will then use a “M2” metatarsal implant with a “P1” phalangeal implant, as well as the B2T1 bearing. The “bearings” are the UHMWPE circular components of the implant set, that snap into the base of the phalangeal implant. For the labeling under the UHMWPE bearing, the first number represents the size Phalangeal implant that bearing is used with, while the second number represents which size Metatarsal implant the bearing is meant to be used with. Each “M” and “P” implant can be used with its' corresponding size implant, as well as one size up and one size down with the use of these bridging bearings (except for M1, P1, M5 and P5 which can only be used with its corresponding size and one more). If it is discovered that too much phalangeal bone canal is resected with the 4 mm burr (by inserting the desired reamer into the bone canal), it is recommended that the surgeon use a phalangeal titanium implant that is one size up from the metatarsal implant. For instance, after sizing the patient, if a M4 implant is intended to be used with a P4 titanium implant, the surgeon would then instead use a P5 titanium implant with a B5T4 bearing.
4. The large hand held broche instruments are used to remove a portion of the patient's medullary canal where the stem of the implant will be inserted. The rasp ends of these broche instruments are barely smaller than the implants stems' to prevent reaming a larger canal than needed. The size implants that best correspond to each patient will determine which size broche instruments are to be used to assist in implanting that specific device. The broche instruments are double sided. For example, the “M1” implant will have its canal made with the use of the broche instrument with the end labeled “M1”, and the “P1” implant will have its canal reamed with the rasp end labeled “P1”. The Metatarsal side of each broche instrument is made at the same declination angle that the “M” implants heads sit on their stems, which is 15 degrees on the horizontal axis. When reaming out each bone canal, insure that the reamer handle markings are oriented dorsally (vertically at 180 degrees relative to the transverse plane).
5. After the appropriate implants are chosen, take the selected titanium phalangeal implant and insert it into the bearing seater instrument. Then, take the selected corresponding UHMWPE bearing and place it on top of the phalangeal tray. Next, take the phalangeal impactor instrument and use a standard orthopedic mallet to tap and seat the UHMWPE bearing into the titanium phalangeal implant.
6. After seating the bearing into the phalangeal implant, take the selected metatarsal implant, and after using the desired amount of bone cement, use the metatarsal impactor instrument and mallet to insert the metatarsal implant into the medullary canal of the first metatarsal. Upon implanting the metatarsal implant, insure that the ridge on the head is placed vertically (at 180 degrees) relative to the transverse plane.
7. After implanting the metatarsal component, use the desired amount of bone cement, then use the phalangeal impactor instrument and mallet to insert the assembled phalangeal implant into the medullary canal of the proximal phalanx. If the great toe is not anatomically aligned, it is important that the EHL (extensor hallucis longus) be lengthened to maintain great toe anatomic alignment. It is also important that the tendon sheath/paratenon of the EHL be sutured to 1^{st} MPJ the joint capsule to keep the EHL in anatomic alignment.
8. After thorough irrigation of the wound, standard bunionectomy closure is performed. An illustration of how the implants look once inserted into the bones is included above.

Additionally, if needed this method of manufacture could be used in the veterinary field on animals.

In certain embodiments, the present invention is used to produce superior total joint replacement systems/implants but partial joint replacement implants as well.

Having thus described several embodiments for practicing the inventive method, its advantages and objectives can be easily understood. Variations from the description above may and can be made by one skilled in the art without departing from the scope of the invention.

The illustrations and images included in this application are of a total joint system implant patented using this method in U.S. Ser. No. 29/611,923, with a filing date of Jul. 26, 2017.

Accordingly, this invention is not to be limited by the embodiments as described, which are given by way of example only and not by way of limitation.

### Lattice Structure

In certain embodiments, the implant includes a lattice structure. The lattice structure allows for the implant to be strong, yet lightweight. Moreover the lattice structure promotes bone regeneration.

### Ridge and Groove

In certain embodiments, a ridge and groove structure are provided in the implant, such that the head of the joint replacement devices for the 1st MTP joint, the lesser MTP joints, and the elbow, have a ridge and groove, such as the ridge that manipulates the sagittal head called the “crista” on a 1st MTP, for added stability of each of these artificial joint replacement, and a corresponding “groove” on the phalangeal side of a 1st MTP total joint replacement, or on any of the above mentioned human joints that were to be constructed with the ridge. The ridge has a corresponding groove to receive it.

### Porous Structure

In certain embodiments, the head of the implant (including but not limited to where the back side of the implant head meets the stem) is porous or has a lattice structure made via 3D printing or other manufacturing.

In certain embodiments, the lattice structure for the implant applies to the following joints: 1st MPJ, Lesser MPJ's, Ankle, elbow, proximal metacarpophalangeal's, Trapezio-Metacarpal Thumb, and the proximal interphalangeal joints

### Natural Angle Achieved

In certain embodiments, all for the 1st MPJMTP (metatarsal-phalangeal) total joint, or lesser MTP total joints that captures the natural declination angle of which the phalanx sits at rest relative to the metatarsal bone, which is between 14 and 19 degrees depending on the patent's anatomy. In these embodiments, the implant are design to be ergonomic and mimic the patient's anatomy.

### Bridging Bearings

In certain embodiments, the bridging bearing non-metallic cap/bearing set of bearings to be used implement technology are referred “bridging bearings”, which means that some of the bearings would bridges the gap between one sized metatarsal implant and a different sized phalangeal implant such as for the 1st Metatarsal-phalangeal Joint, Lesser MPJ's, but also to be used in total Ankle, elbow, proximal metacarpophalangeal's, Trapezio-Metacarpal Thumb, and the proximal interphalangeal joints.

### Disc-Like Caps

In certain embodiments, the implant includes a plurality of disc-like caps, cups or bearings, each size implant's collar having a central opening of the same size and shape so that each cup/cap/bridging bearing for that size implant can universally fits into its respective implant.

### SOURCES CITED

Office of Classification Support. “Class 700 DATA PROCESSING: GENERIC CONTROL SYSTEMS OR SPECIFIC APPLICATIONS.” United States Patent and Trademark Office, United States Government, 2 Jan. 2018, 00:49:07, www. Uspto.gov/web/patents/classification/uspc700/sched700.htm.

Seitz, William H. and Albert N Santilli. “Small joint orthopedic implants and their manufacture. U.S. Pat. No. 8,353,965.

Nutter, David Scott, and Scott Wayne Nutter. “3D Anatomic 1^{st} Metatarsal-Phalangeal Joint Replacement.” U.S. application Ser. No. 29/611,923.

Office of Classification Support. “Nanotechnology.” United States Patent and Trademark Office, United States Government, 2 Jan. 2018, 13:43:47, www.uspto.gov/web/patents/classification/uspc977/defs977.htm#C977S931000.

Office of Classification Support. “Surgery.” Class Schedule for Class 600 SURGERY, United States Government, 8 Feb. 2018, 00:12:46, www.uspto.gov/web/patents/classificaiton/uspc600/sched600.htm.

What is claimed is:

1. A method for manufacturing an anatomic shaped orthopedic joint replacement implant or implants for 1^{st} MPJ, Lesser MPJ's, Ankle, elbow, proximal metacarpophalangeal's, Trapezio-Metacarpal Thumb, and the proximal interphalangeal joints, the method comprising:
determining the location of a joint implant replacement on a patient;
providing an image source from a modeled bone selected from a group consisting of human cadaver bones;
acquiring and producing a three-dimensional (3D) computer file based upon the image source;
using the 3D computer file to create the orthopedic joint replacement implant, wherein the creation of the orthopedic joint replacement implant comprises:
providing an indicator/marking to fabricate the implant, so the implant conforms to the dimensions of the one or more human bones,
providing a stem/anchor to attach or fit on to the collar or head of the implant,
providing a cap or bearing (usually a plastic or polymer of some kind) to fit on to the collar/head of the implant to prevent metal-on-metal contact; and
inserting the implant into the patient via a surgical procedure.

2. The method of claim 1, wherein the 3D computer file alters the image source to erase any imperfections to make smooth surfaces for the orthopedic joint replacement implant.

3. The method of claim 1, wherein the 3D computer file is used with a 3D printer to create the orthopedic joint replacement implant.

4. The method of claim 1, wherein human bones are used as the model for the image source to design the orthopedic joint replacement implant.

5. The method of claim 1, wherein the step of acquiring and producing the 3D computer file includes segregating the part of the bone where the joint resurfacing most commonly starts and saving the part of the bone into a new computer file.

6. The method of claim 1, wherein the head of the joint replacement devices for the 1st MTP hemi or total joint, the lesser MTP joints, and the elbow have a ridge and groove, such as the ridge that manipulates the sagittal head called the “crista” on a 1^{st} MTP, for added stability of each of these artificial joint replacement, and a corresponding “groove” on the phalangeal side of a 1^{st} MTP total joint replacement, or on any of the above mentioned human joints that were to be constructed with a ridge to have corresponding groove to receive it.

7. The method of claim 1, wherein parts of or all of the implants' stem is porous, or has a lattice structure made via 3D printing or other manufacturing methods to be implemented in the following joints: 1^{st} MPJ, Lesser MPJ's, Ankle, elbow, proximal metacarpophalangeal's, Trapezio-Metacarpal Thumb, and the proximal interphalangeal joints.

8. The method of claim 1, further comprising altering the articulating surfaces of the modeled bone in the 3D file, such as its cortical surface or surfaces, to erase any imperfections or deviations from the natural obvious contours of the one or more cadaver bones.

9. The method of claim 1, wherein the one or more bones are cadaver bones.

10. The method of claim 1, wherein the one or more bones are non-living human bones.

11. The method of claim 1, wherein the surgical procedure is an osteotomy.

12. The method of claim 11, wherein the osteotomy performed uses a 1^{st} MTP total joint replacement device that captures the natural declination angle of which the phalanx sits at rest relative to the metatarsal bone, which is between 14 and 19 degrees depending on the patent's anatomy.

13. An anatomic shaped orthopedic joint replacement implant produced via the method of claim 1, the anatomic shaped orthopedic joint replacement implant comprising:
a stem/anchor to attach or fit on to the collar or head of the implant; and
a polymer/plastic or non-metallic cap/bearing to fit on to the collar/head of the implant.

14. The anatomic shaped orthopedic joint replacement implant produced via the method of claim 13, wherein the non-metallic cap/bearing bridges the gap between one sized metatarsal implant and a different sized phalangeal implant, 1^{st} MPJ, Lesser MPJ's, Ankle, elbow, proximal metacarpophalangeal's, Trapezio-Metacarpal Thumb, and the proximal interphalangeal joints.

15. The orthopedic joint replacement implant produced via the method of claim 14, further comprising a plurality of disc-like caps, cups or bearings, each size implant's collar having a central opening of the same size and shape so that each cup/cap/bridging bearing for that size implant universally fits into its respective implant.

16. The method of claim 15, wherein the head of the implant is porous or has a lattice structure made via 3D printing or other manufacturing methods to be implemented in the following joints: 1^{st} MPJ, Lesser MPJ's, Ankle, elbow, proximal metacarpophalangeal's, Trapezio-Metacarpal Thumb, and the proximal interphalangeal joints.

17. An Anatomic shaped orthopedic joint replacement implant produced via the method of claim 1, wherein the osteotomy performed uses a 1^{st} MTP total joint replacement device that captures the natural declination angle of which the proximal phalanx sits at rest relative to the metatarsal bone, which is between 14 and 19 degrees.

18. An anatomic shaped orthopedic joint replacement implant, wherein the head of the joint replacement device for a 1st MTP hemi or total joint, the lesser MTP joints, and the elbow have a ridge and groove, such as the ridge that manipulates the sagittal head called the “crista” on a 1st MTP, for added stability of each of these artificial joint replacement, and a corresponding “groove” on the phalangeal side of a 1st MTP total joint replacement, or on any of the above mentioned joints that would be constructed with a ridge to have corresponding groove to receive it.

19. An anatomic shaped orthopedic joint replacement implant wherein parts of or all of the implants' head or stem is porous, or includes a porous or lattice structure made via 3D printing or by means of other manufacturing methods, to be implemented in the following joints: 1st MPJ, Lesser MPJ's, Ankle, elbow, proximal metacarpophalangeal's, Trapezio-Metacarpal Thumb, and the proximal interphalangeal joints.

20. An anatomic shaped orthopedic joint replacement implant wherein the cap/bearing set of bearings to be used implement technology we refer to as “bridging bearings”, wherein such a bearing bridges the gap between one sized metatarsal implant and a different sized phalangeal implant, such as for the 1st Metatarsal-phalangeal Joint, Lesser MPJ's, but also to be used in bridging the gap between different sized implants for the total Ankle, elbow, proximal metacarpophalangeal's, Trapezio-Metacarpal Thumb, and the proximal interphalangeal joints to allow mix and match of different sized implants together.
